(19) United States
(12) Patent Application Publication
(10) Pub. No.: US 2002/0177546 A1
(43) Pub. Date: Nov. 28, 2002

(54) Dual therapy method of treating and controlling diabetes mellitus

(76) Inventors: W. Blair Geho, Wooster, OH (US); Hans C. Geho, Solon, OH (US)

Correspondence Address: SDG, INC., HANS C. GEHO, 1350 EUCLID AVENUE, SUITE 200, CLEVELAND, OH, 44115, US

(21) Appl. No.: 09/828,077

(22) Filed: Apr. 6, 2001

(51) Int. Cl. A61K 38/28; A61K 31/4045

(52) U.S. Cl. 514/3; 514/415

(57) ABSTRACT

A dual therapy method of preventing, treating, and controlling diabetes mellitus complications is disclosed. The method comprises simultaneously treating a patient in need thereof with an effective amount of a mixture of insulin and serotonin contained in a Hepatocyte Directed Delivery system.

### BACKGROUND OF THE INVENTION

1. Field of the Invention

This invention relates to a dual therapy method of preventing, treating or controlling diabetes mellitus, and, more particularly, by preventing, treating or controlling such disease and the complications associated therewith with a simultaneous delivery of insulin and serotonin.

2. Description of the Related Art

Diabetes mellitus has been defined as 1) a random or casual plasma glucose >200 mg/dL (11.1 mmol/L), associated with symptoms (such as, but not limited to, polyuria, polydypsia, unexplained weight loss); 2) fasting plasma glucose 126 mg/dL (7.0 mmol/L); or 3) a 2-hour glucose reading of >200 mg/dL (11.1 mmol/L) after a 75-gram glucose load. Any of these three are sufficient for a diagnosis, but each should be confirmed on a separate day.

It has been previously established that, in the fed state, normal carbohydrate metabolism requires hepatic uptake and storage of glucose triggered by the delivery of insulin and serotonin (see U.S. Pat. Nos. 4,603,044; 4,704,394; and 4,761,287). Without both hormones acting upon the liver, normal glucose metabolism does not occur, and the patient experiences the diabetic state of hyperglycemia. It has been previously shown that diabetic patients having a paucity of endogenous hepatic insulin but an adequate supply of endogenous hepatic serotonin can be treated with a combined form dose of hepatic-targeted insulin and free (hepatic-targeted) insulin (see U.S. Pat. No. 4,863,896). Conversely, it has also previously been shown that diabetic patients having a paucity of endogenous hepatic insulin but an adequate supply of endogenous hepatic serotonin can be treated with a combined form dose of hepatic-targeted insulin and free (non-hepatic-targeted) insulin (see U.S. Pat. No. 4,863,896). Conversely, it has also been previously shown that diabetic patients having a paucity of endogenous serotonin but an adequate supply of endogenous hepatic insulin can be treated with hepatic-targeted serotonin (see U.S. Pat. No. 4,761,287).

Concurrently, type 1 diabetic patients are treated by the administration of insulin, primarily by subcutaneous injection. Type 2 diabetic patients are generally believed to be insulin resistant, and most current type 2 therapies are based on this belief. Typically, the first method of treatment for type 2 diabetes patients is to control diet and weight and to exercise. When this method of treatment no longer provides adequate glycemic control, patients are placed on a variety of oral anti-diabetic medications such as sulfonyl ureas, metformin, glucosidase inhibitors, and triglitizone derivatives. The actions of these drugs may vary, but their net result is to enhance the action of endogenous insulin. Again, the rationale for these therapies is based on the theory of insulin resistance, i.e., either an excess of insulin is required to overcome this resistance or the cellular response to insulin must be directly modified.

### SUMMARY OF THE INVENTION

This invention relates to a method of controlling or treating diabetes mellitus, and, more particularly, by treating such disease and the complications associated therewith with a simulataneous delivery of insulin and serotonin. In particular, the simultaneous delivery is carried out by means of a hepatocyte directed delivery system.

### DETAILED DESCRIPTION OF THE INVENTION

The present invention relates to a method of controlling or treating diabetes mellitus in a mammal, e.g., a human being or an animal. The method comprises the simultaneous administration to such patient in need thereof an effective amount of a mixture of insulin and serotonin.

It is believed that diabetes mellitus patients usually begin with a discreet type of the disease. That is, it is generally held that diabetic patients are either insulin-deficient (type 1) or insulin resistant (type 2). The 1997 Expert Committee on the Diagnosis and Classification of Diabetes Mellitus also recognized a third class and a fourth class of diabetes mellitus, which classes are described in Diabetes Care 20:1183, 1997. Patients in these latter two classes usually exhibit some form and/or degree of insulin deficiency or insulin resistance. It is also the inventors' belief that diabetic patients who have normal or elevated levels of insulin but who may be insulin resistant (and who perhaps may ultimately require insulin therapy) have serotonin-deficient diabetes. Insulin resistance, if it occurs in a serotonin-deficient patient, is possibly a result of long-term diabetes and current methods of treatment. Accordingly, for the purpose of this invention and the disclosure contained herein, references to serotonin-deficient diabetes mellitus will also be presumed to include type 2 diabetes mellitus, as well as other emerging classes of diabetes that are not due exclusively to the absence or lack of insulin. Conversely, for the purpose of this invention and the disclosure contained herein, references to type 2 diabetes mellitus and other emerging classes of diabetes that are not due exclusively to the absence or lack of insulin will be presumed to include serotonin-deficient diabetes mellitus.

It is the new belief that over time, many patients having discreet forms of either type 1 or type 2 diabetes develop a combined disease of type 1 and type 2 diabetes. In other words, they lose both their endogenous insulin and their endogenous serotonin. Currently, this class of patients is presently treated as though they are type 1 diabetes patients because the disclosure contained herein of serotonin deficiency has not as yet been disclosed in the medical literature.

In recognition of the fact that certain patients develop both types of diabetes, the invention herein disclosed provides combined insulin and serotonin therapy for diabetic patients evidencing a lack of hepatic insulin and serotonin. In such patients, insulin and serotonin must be administered prior to ingestion of a meal and/or as a basal, or fasting, dose and must be delivered simultaneously to the liver and, specifically, to the hepatocytes. Such delivery will convert the liver from net glucose output to net glucose uptake during ingestion and absorption of a meal.

The term “insulin” shall be interpreted to encompass natural extracted human insulin, recombinantly produced human insulin, insulin extracted from bovine and/or porcine sources, recombinantly produced bovine and/or porcine insulin and mixtures of any of these insulin products. The term is intended to encompass the polypeptide normally used in the treatment of diabetics in a substantially purified form but encompasses the use of the term in its commercially available pharmaceutical forms, which includes analogs and additional excipients. The term also includes both short-acting and long-acting insulins. The insulin is preferably recombinantly produced and may be dehydrated (completely dried) or in solution.

The terms “insulin analog,” “monomeric insulin” and the like are used interchangeably herein and are intended to encompass any form of “insulin” as defined above wherein one or more of the amino acids within the polypeptide chain has been replaced with an alternative amino acid and/or wherein one or more of the amino acids has been deleted or wherein one or more additional amino acids has been added to the polypeptide chain or amino acid sequences which act as insulin in decreasing blood glucose levels. In general, the “insulin analogs” of the present invention include “insulin lispro analogs,” as disclosed in U.S. Pat. No. 5,547,929, incorporated hereinto in its entirety by reference, insulin analogs including Lispro insulin and humalog insulin, and other “super insulin analogs,” wherein the ability of the insulin analog to affect serum glucose levels is substantially enhanced as compared with conventional insulin as well has hepatoselective insulin analogs which are more active in the liver than in fat and/or adipose tissues. Preferred analogs are monomeric insulin analogs, which are insulin-like compounds used for the same general purpose as insulin such as insulin lispro, i.e., compounds which are administered to reduce blood glucose levels.

For convenience purposes herein only, whenever the term “insulin” is employed, it shall encompass any of an “insulin,” an “insulin analogue,” or a “monomeric insulin” agent as they are defined above.

The term “serotonin” shall be interpreted to mean 5-hydroxytryptamine (“5-HT”) and it also includes but is not limited to any and all “serotonin analogs” which are defined as agents that act as agonists at hepatic 5-HT receptors and/or their subtypes, including but not limited to those 5-HT receptors identified as 5-HT1A, 5-HT1B, 5-HT1C, 5-HT1D, and all other 5-HT1 receptor subtypes, 5-HT2A, 5-HT2B, 5-HT2C, 5-HT2D, and all other 5-HT2 receptor subtypes, 5-HT3 and all 5-HT3 receptor subtypes, 5-HT4 and all 5-HT4 receptor subtypes, 5-HT5 and all 5-HT5 receptor subtypes, 5-HT6 and all 5-HT6 receptor subtypes, and 5-HT7 and all 5-HT7 receptor subtypes.

In addition, for the purpose of this disclosure, “serotonin analogs” will also be defined as any agent that either acts as an agonist at hepatic serotonin receptors (i.e., “serotonergic agent”) or potentiates the activity of serotonin or serotonergic agents at hepatic serotonin receptors, including, but not limited to, agents of the following classes:

| | |
|---|---|
| Class 1: | Serotonin precursors including, but not limited to, tryptophan. |
| Class 2: | Serotonin analogs that are of a class whose activity are blocked |
| | by serotonin receptor antagonists such as, but not limited to, |
| | methysergide, ketanserin, clozapine, risperidone, and |
| | cyproheptadine. |
| Class 3: | Serotonin receptor agonists such as, but not limited to, |
| | buspirone, ergot alkaloids, sumatriptan, cisapride, D-Lysergic |
| | Acid Diethylamide (“LSD”), 8-Hydroxy-(2-N,N-Dipropyl- |
| | amino)-Tetraline (8-OH-DPAT), and m-Chlorophenylpiperazine |
| | (“mCPP”). |
| Class 4: | Agents that block the reuptake of serotonin, including, but not |
| | limited to, monoamine oxidase inhibitors (“MAOI”) such as |
| | phenelizine, tranylcypromine, selegiline, and isocarboxacid; and |
| | tricyclid antidepressants such as amitryptiline, amoxapine, |
| | clomipramine, desipramine, dexopin, imipramine, maprotiline, |
| | nortryptiline, protryptiline, and trimipramine. |
| Class 5: | Agents that selectively inhibit serotonin reuptake, also known as |
| | selective serotonin reuptake inhibitors (“SSRI”), including, but |
| | not limited to, fluoxetine, sertraline, paroxetine, and |
| | fluvoxamine. |
| Class 6: | Agents that enhance serotonin release, including, but not limited |
| | to, d-amphetamine, reserpine, methylphenidate, and pemoline. |
| Class 7: | Over the years, there have been a number of agents discovered |
| | that are clearly classed as serotonin receptor agonists, serotonin |
| | reuptake inhibitors, SSRIs, MAOIs, or serotonin release |
| | enhancers; however, there are other new agents that do not fall |
| | clearly into these identified classes. These agents are defined |
| | as atypical or second generation agents, and include, but are |
| | not limited to buproprion, nefazadone, trazadone, and |
| | venlafaxine. In addition, some metabolites of these agents, |
| | such as m-chlorophenylpiperazine, a metabolite of trazadone, |
| | act as serotonin agonists. Moreover, some of these agents are |
| | pro-drugs that become active only when metabolized. |

The term also includes both short-acting and long-acting serotonins.

For convenience purposes herein only, whenever the term “serotonin” is employed, it shall encompass any of the agents or components of a “serotonin” as it is defined above.

Effective amounts of hepatic-directed insulin and hepatic-directed serotonin can be combined in any conventional manner known in the art, and may, if desired, be further combined with an additional free dose (i.e., not hepatic-directed) of insulin, as described in U.S. Pat. No. 4,863,896; and then such a resultant mixture is given to the patient in such combined dose form orally, or by subcutaneous injection or intravenously, or by nasal or oral inhalation (typically via nebulizer or conventional pressurized aerosol means), by an implated pump device, by topical delivery, or by suppository. However, to assure directed administration to the liver of those active agents that should be hepatically-directed, a preferred method is to deliver a therapeutic mixture comprising hepatic-directed insulin and serotonin by means of a liposomal and/or polymeric hepatocyte-directed delivery system (hereinafter referred to as a “HDD” system).

The preparation of such an HDD system and the incorporation therein of insulin alone in its effective amount and serotonin alone in its effective amount is also well known. In this regard, reference is made to U.S. Pat. Nos. 4,377,567; 4,603,044; 5,104,661; 4,761,287; 4,704,394; and 6,063,400; all of which are incorporated hereinto by reference in their entirety.

The resultant insulin in its HDD system and serotonin in its HDD system are then combined to form an HDD system having a therapeutic mixture of combined insulin and serotonin.

Alternatively, both an effective amount of insulin for treating diabetes mellitus in a patient in need thereof and an effective amount of serotonin for such treatment can be combined to form a mixture which is then incorporated or associated with the HDD to form such an HDD system containing the therapeutic mixture.

In addition to the HDD system composition comprising a therapeutic combination of effective amounts of insulin and serotonin being employed, effective amounts of free insulin and/or free serotonin can be simultaneously administered to the patient along with the HDD system containing the therapeutic mixture as is taught in U.S. Pat. No. 4,863,896, which is incorporated hereinto by reference in its entirety.

The effective amount of insulin in combination with serotonin that is administered will, of course, be dependent on the subject being treated, the type and severity of the affliction, the manner of administration, and the judgment of the prescribing health care provider. Although effective dosage ranges for such specific biologically active substances of interest are dependent upon a variety of factors, and are generally known to one of ordinary skill in the art, some dosage guidelines can be generally defined.

Typically, the insulin is present in the therapeutic mixture or combination in the bottle (pre-administration) in an amount ranging from about 10 Units/ml to about 1000 Units/ml, whereas the serotonin component is present in an amount ranging from about 0.1 mg/ml to about 200 mg/ml. Preferrably, the effective amount to be delivered to the patient in the therapeutic mixture is from about 0.01 Units of insulin to about 10 Units of insulin per kilogram of body weight and from about 0.01 micrograms to about 50 milligrams of serotonin per kilogram of body weight of such patient. Where free insulin and/or free serotonin is also employed, the concentration of these free hormones employed ranges from 1-99% free insulin and about 0-25% free serotonin.

The resultant combined forms HDD product may be administered orally, subcutaneously, intravenously, by inhalation, topically, or by suppository prior to each meal. It is believed that prolonged treatment with this product may actually improve long-term glycemic control to the point that doses may be reduced and/or become less frequent. As indicated, the resultant HDD product may also be used along with standard long-acting insulin and serotonin to supply a basal insulin and/or serotonin dose. Ordinarily, the resultant HDD therapeutic mixture, alone or with an additional hormone or hormones, e.g., free insulin/serotonin, should be administered within one hour prior to food ingestion, as indicated in U.S. Pat. No. 4,761,287, which is incorporated hereinto by reference in its entirety.

It is to be noted that when the HDD product is administered subcutaneously, the insulin and/or serotonin may be actually released over the time from the subcutaneous tissue, thus providing a depot-like basal dose in addition to a short-acting effect.

Normalization of the type 1/type 2 diabetic patient's glycemic control with this HDD therapeutic mixture formulation should, in the absence of unrelated factors, re-establish a normal blood glucose, lipid and weight profile and help prevent long-term diabetes mellitus complications, such as chronic hyperglycemia, as well as hypoglycemia, dyslipidemia, microvascular and macrovascular diseases including visual impairment and blindness, nephropathy and renal failure, hypertension, stroke, atherosclerosis, cardiovascular disease, neuropathy, abnormal hemostasis and immune system disorders. Accordingly, these complications can be prevented or ameliorated or at least the onset of these complications can be delayed and even reversed by the administration to the patient of the inventive formulation disclosed herein.

1. A method of treating or controlling diabetes mellitus in a patient in need thereof, which comprises:
treating said patient with an effective amount of a mixture comprising insulin combined with serotonin.

2. The method as defined in claim 1 wherein said insulin and said serotonin are individually added each in effective amounts to a hepatocyte directed delivery system to form said effective amount of said mixture.

3. The method as defined in claim 1 wherein an effective amount of said insulin and an effective amount of said serotonin are each separately and individually contained in a hepatocyte directed delivery system and said resulting systems are combined to form said effective amount of said mixture.

4. The method as defined in claim 1 wherein said mixture comprises an effective amount of insulin which ranges from about 0.01 Units per kilogram of body weight of the patient and serotonin which ranges from about 0.01 micrgrams to about 50 milligrams per kilogram of body weight of the patient.

5. The method as defined in claim 2 wherein non-hepatic directed insulin and/or non-hepatic directed serotonin are individually added each in effective amounts to the hepatic directed delivery system mixture of insulin and serotonin, for distribution to non-hepatic tissues.

6. A method of preventing or treating complications related to diabetes mellitus selected from and related to chronic hyperglycemia, as well as hypoglycemia, dyslipidemia, microvascular and macrovascular disease including visual impairment and blindness, nephropathy and renal failure, hypertension, stroke, atherosclerosis, cardiovascular disease, neuropathy, and abnormal hemostasis and immune system disorders, which comprises simultaneously treating a patient in need thereof with an effective amount of insulin combined with serotonin.

7. The method of claim 6 wherein said insulin and said serotonin are each individually added to a hepatocyte directed delivery system to form said effective amount.

8. The method of claim 6 wherein said insulin and said serotonin are each individually contained in a separate hepatocyte directed delivery system and said resulting systems are combined to form said effective amount.

9. The method as defined in claim 6 wherein non-hepatic directed insulin and/or non-hepatic directed serotonin are individually added each in effective amounts to a hepatic directed delivery system mixture of insulin and serotonin, for distribution to non-hepatic tissues.

10. The method as defined in claim 4 wherein said effective amount ranges from about 0.01 to about 10 Units per kilogram of body weight of the patient of insulin and from about 0.01 micrograms to about 50 milligrams per kilogram, of body weight of the patient of serotonin.

11. An article of manufacture which comprises:
(a) a hepatocyte directed delivery system (“HDD”)
(b) a mixture of an insulin and a serotonin inculcated thereinto and/or thereupon.

12. An article of manufacture as defined in claim 11 wherein non-hepatic directed insulin and/or non-hepatic directed serotonin are individually added each in effective amounts to a hepatic directed delivery system mixture of insulin and serotonin, for distribution to non-hepatic tissues.
